(19) United States
(12) Patent Application Publication
(10) Pub. No.: US 2025/0090111 A1
(43) Pub. Date: Mar. 20, 2025

(54) SYSTEM AND METHODS FOR CONTROL OF MOTION-ASSISTED TABLE

(71) Applicant: GE Precision Healthcare LLC, Waukesha, WI (US)

(72) Inventors: Mark David Schmieding, Pewaukee, WI (US); Andrew Moulton, Waukesha, WI (US); Julie Silvers, Milwaukee, WI (US); Christopher Fang, Brookfield, WI (US); Jeremiah Joswig, Wauwatosa, WI (US); Jiaqi Li, Pewaukee, WI (US)

(21) Appl. No.: 18/471,173

(22) Filed: Sep. 20, 2023

(51) Int. Cl. A61B 6/04; A61B 6/00; A61B 90/00

(52) U.S. Cl. CPC A61B 6/0487; A61B 6/0407; A61B 6/467; A61B 6/54; A61B 2090/064

(57) ABSTRACT

Methods and systems are provided for controlling a patient table. In one example, a system may include a patient table, an imaging system, and a controller. The controller may be configured to adjust a drive wheel output of the patient table responsive to a user force input received at the patient table when undocked from the imaging system, and adjust a vertical height of the patient table responsive to the user force input when docked to the imaging system. For example, only the drive wheel output may be adjusted responsive to the user force input when the patient table is undocked, and only the vertical height of the patient table may be adjusted responsive to the user force input when the patient table is docked. When not docked, vertical and horizontal adjustments may be enabled simultaneously, in one or more examples

### FIELD

The present disclosure relates to systems and methods for control of a motion-assisted table. In one or more examples, the motion-assisted table may be a patient table, and the patient table may be configured to dock to an imaging system.

### BACKGROUND/SUMMARY

Motion-assisted patient tables may provide motor assistance enabling a user to move the table with reduced effort. For example, motion-assisted patient tables may detect an amount of force input by a user to the table via force sensors, and provide motor assistance to a drive wheel of the table based on the force input detected. In this way, a user may push or pull on the motion-assisted patient table and receive a corresponding amount of drive motor assistance.

In addition to providing drive motor assistance to a motion-assisted patient table, it may also be desirable to provide vertical motor assistance for vertical adjustment of the motion-assisted patient table. However, the ability to accurately discern a direction of force via force sensors is limited. Thus, there are challenges in determining whether to provide driving motor assistance along a horizontal axis, or whether to provide vertical motor assistance along a vertical axis based on force sensor detection.

Previous approaches have attempted to address this issue by relying upon a user force input to provide motor assistance along one axis and then a different type of user input device for a second axis. For example, some example approaches have utilized force sensors for driving motor assistance along a horizontal axis and a user input holding buttons down for vertical motor assistance along a vertical axis.

However, the inventors herein have recognized potential issues with such approaches. As one example, hand repositioning to provide different types of user input may lead to inefficient positioning of the table. Approaches requiring user inputs that include holding a button down may be particularly inefficient.

The inventors have thus developed systems and methods to at least partially address the above-recognized issues. In one example, a system in accordance with the present disclosure may comprise a patient table with a controller configured to adjust a drive wheel output of the patient table responsive to a user force input when in an undocked state; and adjust a vertical height of the patient table responsive to the user force input when in the docked state. The patient table may be docked and undocked to an imaging system, for example. In at least one example, a handle of the patient table may comprise a plurality of force sensors configured to detect the user force input.

Thus, the approach developed by the inventors utilizes whether or not the patient table is docked in conjunction with the user force input to a handle of the patient table to provide directionally accurate motor assistance. In one or more examples, the patient table may further be transitioned from the docked state to the undocked state via the user input force to the handle being a pulling force greater than a threshold.

In this way, a user may intuitively provide a force input to the handle of the table and receive directionally accurate motor assistance in pushing, pulling, raising, and lowering of the table.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows a side view of an example motion-assisted table, according to one or more examples of the present disclosure.

FIG. 2A shows a partial view of an example motion-assisted table in an undocked state, according to one or more examples of the present disclosure.

FIG. 2B shows a partial view of an example motion-assisted table in a docked state, according to one or more examples of the present disclosure.

FIG. 3 shows a block diagram of a control system for a motion-assisted table, according to one or more examples of the present disclosure.

FIG. 4A shows a flow chart of an example method for control of a motion-assisted table according to one or more examples of the present disclosure.

FIG. 4B shows a flow chart continuing from the example method of FIG. 4A.

FIG. 5 shows a first example force sensor configuration for a motion-assisted table, according to one or more examples of the present disclosure.

FIG. 6 shows a second example force sensor configuration for a motion-assisted table, according to one or more examples of the present disclosure.

FIG. 7 shows a third example force sensor configuration for a motion-assisted table, according to one or more examples of the present disclosure.

FIG. 8 shows a fourth example force sensor configuration for a motion-assisted table, according to one or more examples of the present disclosure.

### DETAILED DESCRIPTION

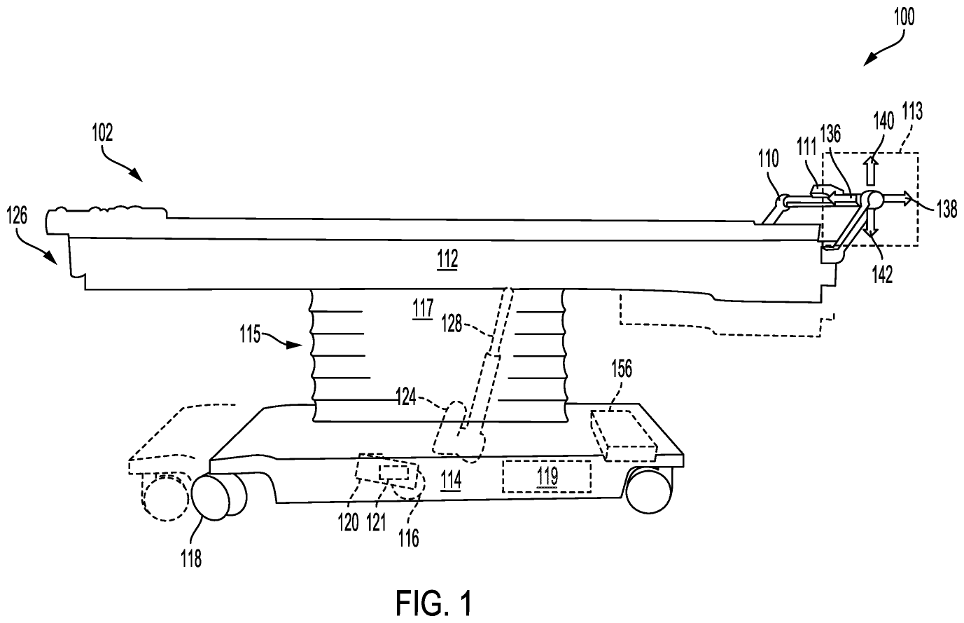

FIGS. 1. 2A, 2B and 5-8 are shown approximately to scale.

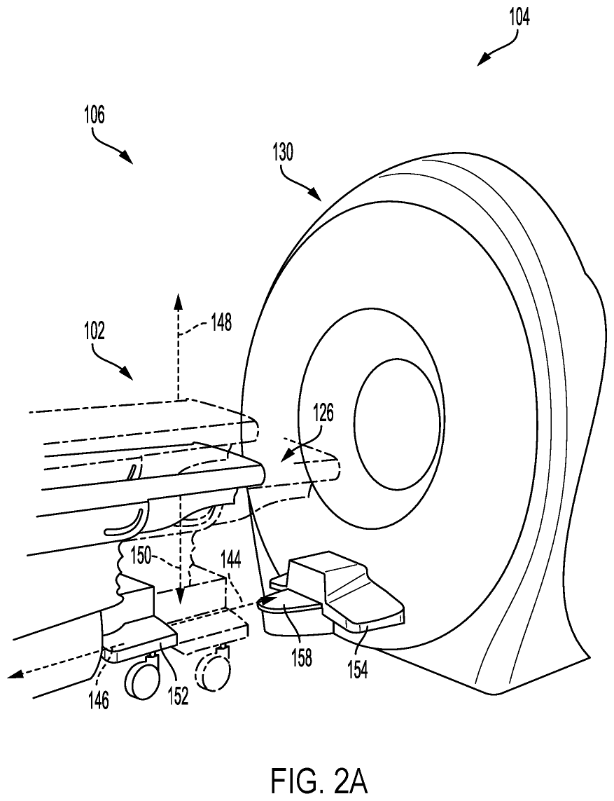

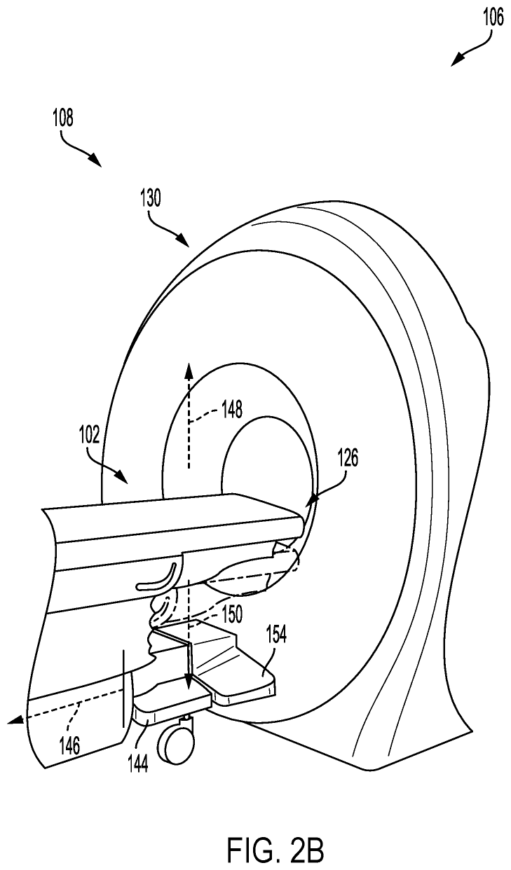

The following description relates to systems and methods for a motion-assisted table, such as the example motion-31 assisted table shown in FIG. 1. The motion-assisted table may be a patient table, in one or more examples. Thus, the motion-assisted table may also be referred to as a patient table or a motion-assisted patient table herein. As shown in the examples at FIGS. 2A and 2B, the patient table may be docked and undocked from an imaging system.

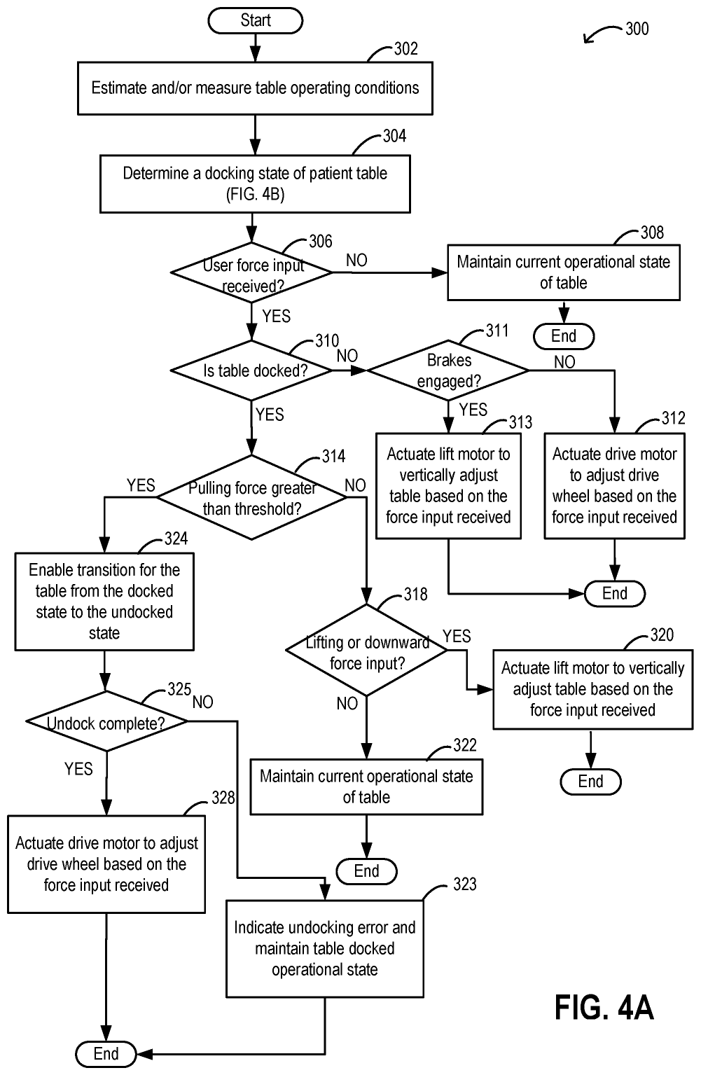

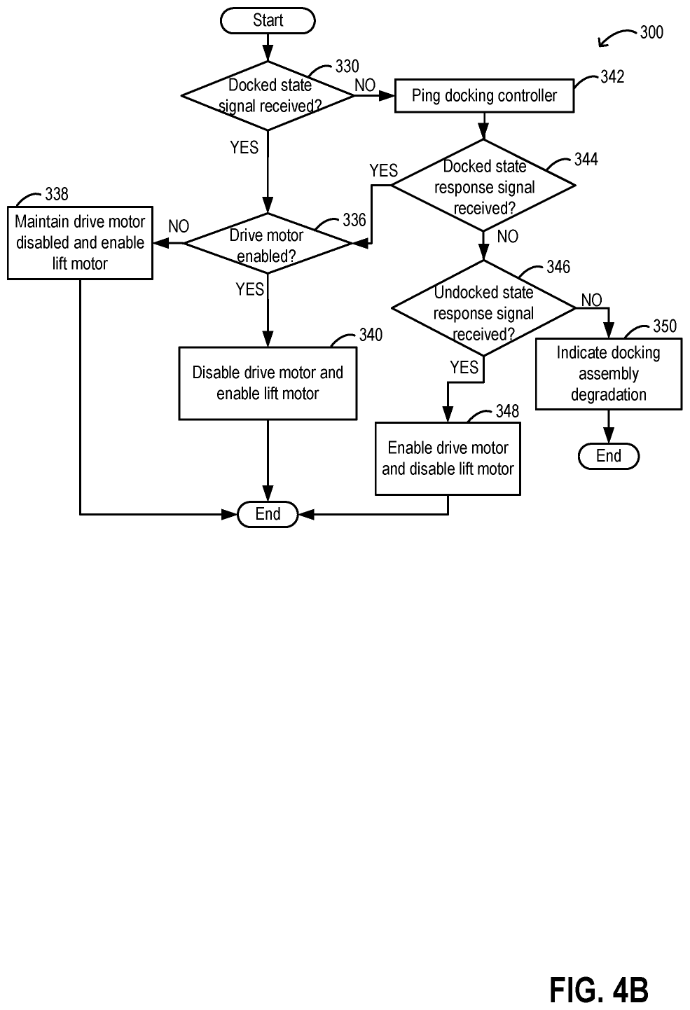

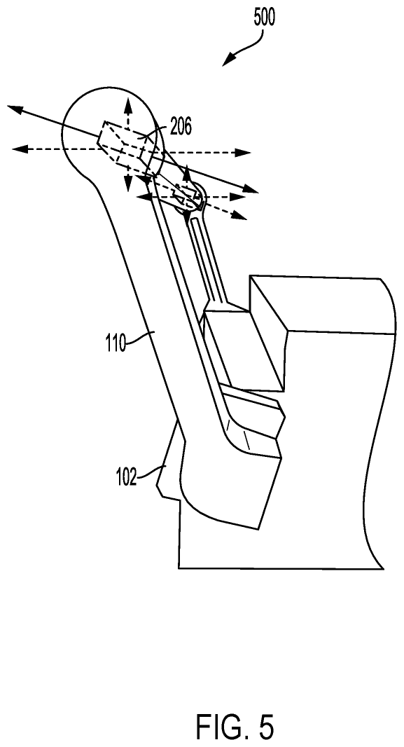

As shown in FIGS. 4A and FIG. 4B, responsive to detecting the patient table is in the docked state, the patient table may be vertically adjusted responsive to a user force input. The user force input may be detected via force sensors. For example, as shown at FIGS. 5-8, a handle of the patient table may comprise a force sensor configuration therein to detect the user force input at the handle.

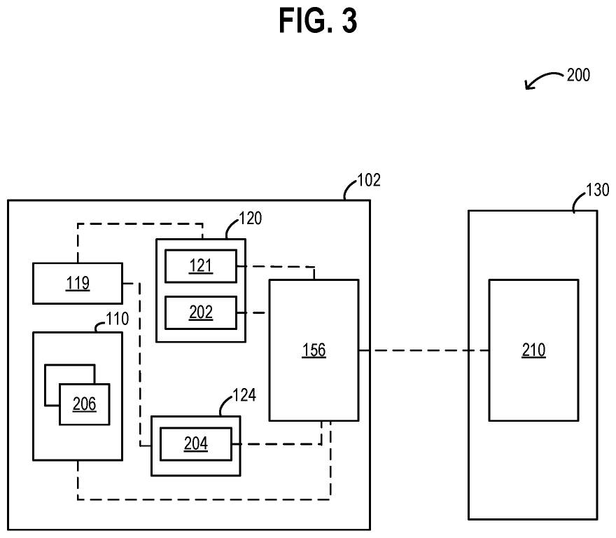

Responsive to detecting the patient table is in the undocked state, the patient table may adjust a drive wheel output of the patient table responsive to the user force input. As shown in FIG. 3, a controller for adjusting the drive wheel output may be separate from the controller for vertically adjusting the patient table. In this way, a user is able to intuitively provide force input to the patient table (e.g., at the handle) to receive automatic motor assistance in adjusting a position of the patient table along a horizontal axis or a vertical axis.

For purposes of discussion, the figures are described collectively. Thus, features that are similar or the same across the figures may be labeled similarly and may not be reintroduced.

Turning now to FIG. 1, a side view 100 of an example motion-assisted table 102 is shown. In particular, side view 100 of FIG. 1 shows a side view of the patient table 102. It is noted that an undocked view 104 at FIG. 2A shows a partial view of the patient table 102 in an undocked state, and docked view 106 of FIG. 2B shows a partial view of the patient table 102 in a docked state.

The motion-assisted table 102 shown in FIG. 1 may be a patient table configured to provide motor assistance responsive to a user input force. As such, the motion-assisted table 102 may also be referred to as a patient table or a motion-assisted patient table herein.

Looking now to side view 100, as may be seen, the patient table 102 comprises a handle 110, a top 112, a base 114, a support 115, and a battery 119, where the battery 119 may be the single and only battery of the patient table 102. The handle 110 comprises a user brake input 111 to engage and disengage brakes 121 of the patient table 102. The handle 110 further comprises force sensors positioned therein. The force sensors may be configured to detect an amount of force, as well as a direction of force input to the handle 110. Further details as to the force sensors that may be provided in the handle 110 are provided at FIGS. 5-8 herein.

As shown at side view 100 of FIG. 1, the patient table 102 may be configured to detect a direction of force input 113 to the handle 110, including whether a pushing force 136, a pulling force 138, a lifting force 140, or a downward force 142 is input at the handle 110. In this way, a direction of the force input at the handle 110 along a first axis and a second axis may be detected. The first axis may be a horizontal axis including the pushing force 136 and pulling force 138, and the second axis may be a vertical axis including the lifting force 140 and downward force 142.

Continuing with side view 104, a drive wheel 116 and a plurality of passive wheels 118 are coupled to the base 114 of the patient table 102. For example, one drive wheel 116 and four passive wheels 118 are included in the example patient table 102 of FIG. 1, where the drive wheel 116 is positioned approximately at a center of the patient table 102 and each of the passive wheels 118 are positioned at a different corner of the table 102. However, it is noted that different wheel configurations are possible. There may be more drive wheels 116 and/or more of fewer passive wheels 118, for example. Additionally, or alternatively, a positioning of the drive wheel 116 and the passive wheels 118 may be varied, in some examples.

The drive wheel 116 may be configured to provide motor assistance to help propel the patient table 102 in a forward or a reverse direction based on the user input force received at the handle 110 of the patient table 102. The forward direction may be a direction towards a foot 126 of the patient table 102, and the reverse direction may be a direction towards the handle 110 of the patient table 102. In this way, the drive wheel 116 is configured to propel the patient table 102 along the first, horizontal axis.

Adjusting the output of the drive wheel 116 via the drive motor 120 may include adjusting one or more of a direction and an amount of torque to output from the drive motor 120 to the drive wheel 116 based on the user force input to the patient table 102. In at least one example, the direction and the amount of torque provided from the drive motor 120 to the drive wheel 116 may be controlled via a corresponding drive motor controller. Further details as to the drive motor controller are discussed at FIG. 3.

Based on the user force input detected via force sensors, during a driving condition, a drive motor 120 coupled to the drive wheel 116 is configured to adjust a torque output of the drive wheel 116. In at least one example, when the user force input at the handle 110 is detected as the pushing force during the driving condition, the drive wheel 116 may be configured to provide motor assistance to help propel the patient table 102 in the forward direction. When the user force input at the handle 110 is detected as the pulling force during the driving condition, the drive wheel 116 may be configured to provide motor assistance in the reverse direction. Further details as to detecting the driving condition and adjusting the drive wheel 116 are provided herein at FIGS. 4A and 4B, for example.

The plurality of passive wheels 118 do not have motors coupled thereto. Rather, the plurality of passive wheels 118 may be swivel caster wheels, for example. The plurality of passive wheels 118 may thus assist in supporting the patient table 102 and allow the user to steer the patient table 102 via a steering force input to the table. In this way, the passive wheels 118 are supporting wheels and allow a user to steer the patient table 102, while the drive wheel 116 is configured to provide motor drive assistance in the forward or the reverse direction.

Looking now to the support 115, the support 115 couples the base 114 to the top 112 of the patient table 102. The support 115 may be configured to vertically adjust the patient table 102 when the table is docked in an upward direction or a downward direction, where the upward direction is a direction towards the top 112 of the patient table 102, and the downward direction is a direction towards the base 114 of the patient table 102.

The support 115 may comprise an arm 128 and a lift motor 124, where the lift motor 124 is configured to vertically adjust the arm 128. The arm 128 of the support 115 may be directly coupled to the top 112 and the base 114 of the patient table 102. In some examples, the arm 128 may be a telescoping arm that is extended and retracted via the lift motor 124. When the arm 128 is extended, the top 112 of the patient table 102 may be lifted vertically in the upward direction, and when the arm 128 is retracted, the top 112 of the patient table 102 may be lowered vertically in the downward direction. The top 112 of the patient table 102 may be a patient cradle that is configured to receive and support a patient. Thus, vertical adjustment of the patient table 102 to move the top 112 up and down may be used to position the patient as desired.

In order to extend and retract the arm 128, the arm 128 may be coupled to the lift motor 124 of the support 115, and the lift motor 124 may be configured to adjust torque output to the arm 128 based on the user force input detected via force sensors in the upward direction or the downward direction. The user force input may be a lifting or a downward force input at the handle 110 of the patient table 102. When the user force input at the handle 110 is detected as a lifting force, lift motor 124 may be actuated to lift the patient table 102 in the upward direction. When the user force input at the handle 110 is detected as a downward force, the lift motor 124 may be actuated to lower the patient table 102 in the downward direction. In at least one example, the direction and the amount of torque provided from the lift motor 124 to the support 115 may be controlled via a corresponding lift motor controller. Further details as to the lift motor controller are discussed at FIG. 3.

The support 115 may comprise a housing 117 configured to surround the arm 128 and the lift motor 124, in at least one example. The housing 117 may be configured to extend and collapse with extension and retraction of the arm 128, respectively. Thus, when the arm 128 is extended and the top 112 is lifted vertically in the upward direction, the housing 117 also extends. Further, when the arm 128 is retracted and the top 112 is lowered vertically in the downward direction, the housing 117 may collapse. For example, the housing 117 may comprise an accordion-folded configuration for such extension and collapsing. In some examples, the housing 117 may comprise a flexible material configured to move with the vertical adjustment of the patient table 102. Other possible housing 117 configurations that are able to extend and collapse, such as a telescoping configuration, are also possible.

Turning now to FIG. 2A, the undocked view 104 at FIG. 2A is a partial view of the patient table 102 shown in the undocked state. The undocked view 104 shows the patient table 102 undocked from an imaging system 130. The imaging system 130 may be a medical imaging system such as an MRI system, PET scan system, or X-ray system, among other possibilities. In another representation, it is also possible for the patient table 102 to be docked to a non-imaging system. The patient table 102 at FIG. 2A may be the same or similar as the patient table 102 shown in FIG. 1.

In the undocked state, it is possible for the patient table 102 to provide motor assistance along a first axis in the forward direction 144 and the reverse direction 146, as well as along a second axis in the upward direction 148 and the downward direction 150. The first axis may also be referred to as a horizontal axis and the second axis may be referred to as a vertical axis.

The patient table 102 may be determined as either being in the docked or undocked state via a system level controller 156 of the patient table (e.g., see the system level controller 156 at side view 104). For example, the patient table 102 may comprise a table docking device 152. When the table docking device 152 is engaged with a dock 154 of the imaging system 130, a signal may be output to the system level controller 156 indicating the patient table 102 is in the docked state. Thus, in the absence of a signal indicating the patient table 102 is in the docked state, the system level controller 156 may determine the patient table 102 is in the undocked state.

Further, in some examples, the system level controller 156 may determine the patient table 102 is in the undocked state responsive to one or more of receiving a signal indicating a failure to successfully dock the patient table 102 the imaging system 130, and receiving a signal indicating a release of the patient table 102 from the docked state.

The signal output to the system level controller 156 to indicate the patient table 102 is engaged may be output from one or more sensors of the table docking device 152, in one or more examples. For example, the table docking device 152 may comprise one or more positioning sensors that output a signal to the system level controller 156 responsive to detecting the table docking device 152 is engaged with the dock 154 of the imaging system 130. These positioning sensors may include RF sensors or other sensors capable of detecting such relative positioning.

The dock 154 of the imaging system 130 may comprise an extension 158 configured to mechanically mate with the table docking device 152 of the patient table 102. For example, the extension 158 of the dock 154 may latch into the table docking device 152 of the patient table 102. Additionally, or alternatively, a signal may be output from a docking controller to the system level controller 156, indicating to the patient table 102 whether or not the patient table 102 is in the docked state. The docking controller may be a controller of the imaging system 130, in some examples. In other examples, the docking controller may be a controller included in the table docking device 152.

In at least one example, a dead-man switch in the handle 110 may allow motion along the horizontal axis and the vertical axis at all times, other than when docked to the imaging system 130. For example, the dead-man switch may be a switch lever integrated with the handle 110 such that when an operator grasps the handle 110, motion of the patient table 102 is permitted, and when the operator releases the handle 110, the handle motion is prevented. The type of motion (e.g., vertical or horizontal) may be based on a state of the patient table 102.

Turning now to FIG. 2B, the docked view 106 at FIG. 2B shows the patient table 102 in the docked state. The patient table 102 at FIG. 2B may be similar or the same as the patient table 102 shown at FIG. 1 and/or similar or the same as the patient table 102 shown in FIG. 2A.

As may be seen at FIG. 2B, once docked, vertical motion and the ability to undock the patient table 102 is enabled. However, the ability to move the patient table 102 in the forward direction is disabled in the docked state. That is, in the docked state, it is possible for the patient table 102 to provide motor assistance along the second, vertical axis in the upward direction 148 and the downward direction 150. Further, in the docked state, it is possible for the patient table 102 to be undocked responsive to the user input force at the handle 110 being a pulling force 138 in the reverse direction 146 that is greater than a threshold amount of force.

Moving now to FIG. 3, FIG. 3 shows a block diagram 200 of a control system for a motion-assisted table, such as a motion-assisted table that is the same or similar to patient table 102.

As may be seen at FIG. 3, the system level controller 156 of the patient table 102 may communicate with multiple controllers and the plurality of force sensors 206 in the handle 110 of the patient table 102 to provide motor assistance responsive to the user force input at the handle 110. In particular, the system level controller 156 receives signals from various sensors and controllers, and then the system level controller 156 employs the actuators of FIG. 1 to adjust operation of the patient table 102 based on the received signals and instructions stored on a memory of the system level controller 156.

As illustrated in FIG. 3, the patient table 102 may comprise a separate drive motor controller 202 and lift motor controller 204 that are each controlled based on communication with the system level controller 156 of the patient table 102. In particular, the system level controller 156 of the patient table 102 is configured to receive signals from one or more of the plurality of force sensors 206 and a docking controller 210. Then, based on the signals from the plurality of force sensors 206 and the docking controller 210, the system level controller 156 provides outputs to actuate one or more of the drive motor 120, the brakes 121, and the lift motor 124 of the patient table 102.

In particular, the drive motor 120 and the brakes 121 of the patient table 102 may be actuated via the drive motor controller 202 based on outputs from the system level controller 156, where the drive motor controller 202 may be tuned to actuate the drive motor 120. Based on output from the system level controller 156, the drive motor controller 202 may then adjust operation of the drive motor 120 accordingly. Similarly, the lift motor 124 may be controlled via the lift motor controller 204, where the lift motor controller 204 is tuned to actuate the lift motor 124 based on output from the system level controller 156.

As shown in FIG. 3, the patient table 102 may further comprise a battery 119. In one or more examples, the battery 119 may be the single and only battery of the patient table 102. The battery 119 may be controlled via the system level controller 156 to only provide power to one of the drive motor 120 and the lift motor 124 at a time. Thus, during the undocked state, the battery 119 may be controlled by the system level controller 156 to only provide power to the drive motor 120, and the battery 119 may be configured to not provide power to the lift motor 124 during the undocked state. On the other hand, during the docked state, the battery 119 may be controlled by the system level controller 156 to only provide power to the lift motor 124, and the battery 119 may be configured to not provide power to the drive motor 120 in the docked state. Additionally, or alternatively, power may be provided by the imaging system to the patient table 102 when the patient table 102 is in the docked state.

In this way, based on the user force input to the handle 110 and a docking state of the patient table 102 determined based on signals (or lack of signals) received from the docking controller 210, the system level controller 156 communicates with the drive motor controller 202 and/or the lift motor controller 204 to actuate one or more of the drive motor 120, the brakes 121. and the lift motor 124.

Turning now to FIGS. 4A and 4B, a flow chart of a method 300 is shown. Instructions for carrying out method 300 may be executed by a controller based on instructions stored on a memory of the controller and in conjunction with signals received from sensors of the system according to the present disclosure, such as the sensors described above with reference to FIG. 1. FIG. 2A, and FIG. 2B. The controller may be the same or similar to system level controller 156 and may employ actuators of the patient table (e.g., patient table 102) to adjust patient table operation, according to the methods described below.

Step 302 of method 300 comprises estimating and/or measuring table operating conditions. For example, the system level controller of the patient table may receive one or more signals from the force sensors, the drive motor controller, the lift motor controller, and other potential sensors (e.g., speed sensors) to determine current operating conditions of the patient table.

Following step 302, method 300 comprises determining the docking state of the patient table at step 304 as further detailed at FIG. 4B of method 300. Thus, looking briefly to FIG. 4B, step 304 of method 300 may comprise determining whether a docked state signal has been received at step 330. In one or more examples, the docked state signal may be a signal indicating the patient table is successfully docked to the imaging system. The docked state signal may be a signal output by the docking system controller and received at the system level controller. For example, the table docking device may comprise one or more sensors that engage with the dock of the imaging system and provide an output to the docking system controller responsive to successfully docking the patient table to the imaging system. The docking system controller may then be configured to automatically provide the docked state output signal to the system level controller of the patient table.

Responsive to the docked state signal being received at step 330, method 300 proceeds to step 336.

At step 336, method 300 comprises determining whether or not the drive motor is enabled. If the drive motor is not enabled at step 336, then method 300 comprises maintaining the drive motor disabled and enabling the lift motor at step 338.

Looking back to step 336, if the drive motor is enabled at step 336, then method 300 comprises disabling the drive motor and enabling lift motor at step 340. The drive motor may be disabled by not directing power from the battery of the patient table to the drive motor, while the lift motor may be enabled by directing power from the battery of the patient table to the lift motor.

Furthermore, disabling the drive motor and enabling the lift motor at step 336 may additionally, or alternatively, include updating how force sensor outputs are computed to determine a direction of the force request. For example, because the patient table is docked and the drive motor is disabled while the lift motor is enabled at step 340, output from the force sensors responsive to the user force input may only be used to determine raising and lowering motor assistance, unless a pulling force exceeds the threshold for indicating a desire to undock the patient table.

Following step 340, the flow chart at FIG. 4B may end and method 300 may proceed to step 306 at FIG. 4A.

Looking back now to step 330 at FIG. 4B, if the docked state signal is not received at step 330, then method 300 proceeds to ping the docking controller at step 342. For example, the system level controller may send an output to the docking controller requesting a status update as to the docking status.

Following step 342, if the docked state signal is received at step 344, then method 300 moves to step 336 and proceeds as previously discussed herein. If the docked state signal is not received at step 344, however, then at step 346 method 300 comprises determining whether an undocked state response signal is received in response to pinging the docking controller at step 342.

For example, the undocked state response signal may be provided if the table docking device has been most recently determined to successfully undock from the dock of the imaging system. Additionally, or alternatively, the table docking device may be configured to determine the patient table is in the undocked state responsive to determining the one or more sensors of the table docking device are not docked to the imaging system.

If the undocked state response signal is received at step 346, method 300 comprises enabling the drive motor and disabling the lift motor at step 348. The drive motor may be enabled by directing power from the battery of the table from the drive motor to the lift motor, while the lift motor may be disabled by not directing power from the battery of the patient table to the lift motor. Furthermore, enabling the drive motor and disabling the lift motor at step 336 may additionally, or alternatively, include updating how force sensor outputs are computed to determine a direction of the force request.

For example, because the patient table is in the undocked state at step 348 with the drive motor enabled and the lift motor disabled, output from the force sensors responsive to the user force input may only be used to determine forward and reverse drive motor assistance.

It is noted that reference to enabling and disabling of the drive motor and the lift motor may be carried out in a similar manner for later instances discussed herein.

Looking back now to step 346, if the undocked state signal is not received at step 346, at step 350 it may be determined that there is degradation present with one or more of the docking controller and the one or more sensors of the table docking device.

Looking to step 306 at FIG. 4A, method 300 comprises determining whether a user force input has been received. If the user force input has not been received, then method 300 maintains a current operational state of the patient table at step 308. If the user force input has been received at step 306, then method 300 proceeds to step 310. The user force input may be received at the handle of the patient table. For example, the user force input may comprise one or more of pushing, pulling, lifting, or downward force on the handle of the patient table.

At step 310, it is determined whether the patient table is docked. If the patient table is undocked, method 300 includes determining whether or not a request to engage a brake of the patient table is received at step 311. For example, a user input to a user brake input may request engagement of the brake.

If it is determined that engagement of the brake of the patient table is not present at step 311, method 300 may include actuating the drive motor at 312 to adjust the drive wheel based on the user force input received at step 306. Because the patient table is in the undocked state at step 312, the user force input will not be determined as a lifting or a downward force input. Rather, the user force input will be limited to being interpreted as a pushing or a pulling force input.

For example, responsive to the user force input comprising a pushing force input, the drive motor controller may actuate the drive motor to adjust the drive wheel providing forward rotation of the drive wheel. An amount of torque provided to the drive wheel in the forward direction may be based on an amount of user force input. For example, if the user pushes harder on the handle, and thus provides a higher amount of pushing force, the drive wheel may be adjusted to provide more assistance in the forward direction.

In another example, the user force input may be determined to be a pulling force input. In such examples, the drive motor may controller actuate the drive motor responsive to the pulling force input to adjust the drive wheel and provide reverse rotation of the drive wheel. The more pulling force input to the handle, the more motor assistance provided by way of the drive wheel in the reverse direction. There may be an upper limit torque provided via the drive motor when actuating the drive motor to provide motor assistance in the forward direction or reverse direction responsive to a pushing user force input or a pulling user force input at the handle. The upper limit torque may be based upon a predetermined maximum amount of speed the drive motor may propel the patient table, for example. The upper limit torque for the forward direction may be different than the upper limit torque for the reverse direction, in one or more examples.

However, in one or more representations, it is possible that vertical adjustments and driving adjustments are simultaneously available at step 312. In some examples, the user force input may be used to determine both a lifting force input or downward force input, as well as a pushing force input or pulling force input to simultaneously actuate both a lifting motor and a driving motor accordingly.

Turning back to step 311, if it is determined that a request to engage the brake of the patient table is present at step 311, method 300 may include engaging the brake and actuating the lift motor at 313 to vertically adjust the table based on the user force input received at step 306. Because the patient table is in the undocked state and the brake is engaged at step 313, the user force input will not be determined as a pushing or a pulling force input. Rather, the user force input will be limited to being interpreted as a lifting or a downward force input.

That is, responsive to receiving the user brake request while the patient table is in the undocked state at step 311, method 300 may include engaging the brake of the patient table, preventing the drive assistance for the patient table, and enabling the vertical assistance for the patient table.

For example, responsive to the user force input comprising a lifting force input, the lift motor controller may actuate the lift motor to vertically adjust the table upward to a higher position. An amount of torque provided to the lift motor in the upward direction may be based on an amount of user force input. For example, if the user lifts harder on the handle, and thus provides a higher amount of lifting force, the lift motor may be adjusted to provide more assistance in the upward direction.

In another example, the user force input may be determined to be a downward force input. In such examples, the lift motor controller may actuate the lift motor responsive to the downward force input to adjust the lift motor and provide a lowering of the table. That is, the table may be vertically adjusted downward. The more downward force input to the handle, the more motor assistance provided by way of the lift motor in the downward direction. There may be an upper limit torque provided via the lift motor when actuating the lift motor to provide motor assistance in the upward direction or downward direction responsive to a lifting user force input or a downward user force input at the handle. The upper limit torque may be based upon a predetermined maximum amount of speed the lift motor may vertically adjust the patient table, for example. The upper limit torque for the upward direction may be different than the upper limit torque for the downward direction, in one or more examples.

If the table is in the docked state at step 310, then method 300 includes determining whether a pulling force applied via the user force input is greater than a threshold force. It is noted that the threshold is a non-zero threshold and is indicative that the user desires to transition the patient table from the docked state to the undocked state. The threshold force may be a predetermined force to avoid unintentional transitioning of the table from the docked state to the undocked state. For example, the threshold force may be a pulling force greater than 50 N. In another example, the threshold force may be a pulling force greater than 100 N. Other force ranges may also be possible.

Responsive to the user force input comprising a pulling force greater than the threshold at step 314, method 300 includes enabling the patient table to be transitioned from the docked state to the undocked state at step 324. Enabling transition of the patient table from the docked state to the undocked state may include determining whether the patient table was successfully undocked from the imaging system. For example, the patient table may comprise a latch or other mechanical device coupling the patient table to the imaging system and thus successfully undocking the table may include disengaging the latch or other mechanical device. Additionally, or alternatively, undocking the patient table may comprise electronically releasing the patient table from the imaging system. For example, the imaging system may be configured to operate responsive to the patient table being coupled to the imaging system. Thus, determining whether the patient table was successfully undocked may include electronically undocking the patient table from the imaging system. Following step 325, if the undocking was successful, method 300 may comprise actuating the drive motor to adjust the drive wheel output based on the user force input received at 328.

For example, actuating the drive motor to adjust the drive wheel output based on the user force input received at 328 may include determining whether the user force input is a pushing or pulling force input received at the handle of the patient table. Responsive to the user force input comprising a pushing force input, the drive motor controller may actuate the drive motor to adjust the drive wheel providing forward rotation of the drive wheel. An amount of torque provided to the drive wheel in the forward direction may be based on an amount of user force input. For example, if the user pushes harder on the handle, and thus provides a higher amount of pushing force, the drive wheel may be adjusted to provide more assistance in the forward direction.

In another example, the user force input may be determined to be a pulling force input. In such examples, the drive motor may actuate the drive motor responsive to the pulling force input to adjust the drive wheel and provide reverse rotation of the drive wheel. The more pulling force input to the handle, the more motor assistance provided by way of the drive wheel in the reverse direction.

In some examples, there may be an upper limit threshold for the amount of torque provided via the drive wheel. The upper limit threshold may be based on an upper limit speed for moving the patient table, for example. As one example, the upper limit threshold for the amount of torque provided via the drive wheel may be an amount of torque to move the patient table at a predetermined speed without assistance from the user. Such a predetermined speed may be 15 miles per hour, for example.

Further, transitioning the patient table from the docked state to the undocked state may further include updating how force sensor outputs are computed to determine a direction of the user force input as previously discussed herein.

If the undocking was unsuccessful at step 325, then an undocking error indication may be provided and operation of the patient table may be maintained at step 323. The undocking may be determined as unsuccessful responsive to a failure to transition the patient table from the docked state to the undocked state within a predetermined threshold period of time from the user indication to undock the table at step 314. Additionally, or alternatively, the undocking may be determined as unsuccessful responsive to an output received from the imaging system. The undocking error indication may comprise one or more of a visual indication, such as a light or message on a display device, and an audible alert.

Looking again to step 314, following step 314, method 300 includes determining whether the user input force includes a lifting force or a downward force to the handle at step 318. If not, then the current operational state of the patient table is maintained at step 322.

If the user input force is determined to include a lifting force or a downward force to the handle at step 320, then method 300 includes actuating the lift motor to vertically adjust the patient table based on the user force input received.

For example, actuating the lift motor to vertically adjust the patient table based on the user force input received may include determining whether the user force input is a lifting or downward force input received at the handle of the patient table. Responsive to the user force input comprising a lifting force input, the lift motor controller may actuate the lift motor to adjust the vertical height of the patient table. An amount of torque provided to the lift motor to extend the arm of the support and raise the table vertically may be based on an amount of user force input. For example, if the user lifts up harder on the handle, and thus provides a higher amount of lifting force, the lift motor may be adjusted to provide more assistance in the upward direction. If the user lifts up on the handle with less force, the lift motor may be adjusted to provide less assistance in the upward direction. The one or more force sensors are further configured to detect when the user varies the amount of force input to the handle in a same force direction and correspondingly adjust the amount of torque provided to the lift motor. In this way, the lift motor is responsive to variations in the amount of lifting force on the handle to provide corresponding amounts of motor assistance via the lift motor in the upward direction.

In another example, the user force input may be determined to be a downward force input. In such examples, the lift motor may actuate the arm of the support responsive to the downward force input to lower the table vertically down. The more downward force input to the handle, the more motor assistance provided by way of the lift motor in the downward direction.

In some examples, there may be an upper limit threshold for the amount of torque provided via the lift motor. The upper limit threshold may be based on an upper limit speed for moving the patient table, for example.

Via the example methods and systems disclosed herein, a user is able to receive driving and lifting motor assistance by intuitively inputting force into the handle of the patient table. In particular, lifting, lowering, pushing, and pulling motor assistance are provided in an efficient in and accurate manner, as well as docking and undocking of the patient table.

Turning now to FIG. 5, FIG. 5 shows a first example force sensor configuration 500 for a motion-assisted table, such as a table similar to or the same as patient table 102. Each of the example force sensor configurations shown at FIGS. 5-8 may be separately implemented in different patient tables. Further, each of the example force sensor configurations shown at FIGS. 5-8 may enable a vertical force and a horizontal force of a user force input to be determined independently.

As shown, the first example force sensor configuration 500 comprise force sensors 206 positioned in a handle 110 of the patient table 102. The force sensors 206 may be load cells, in one or more examples. Thus, the force sensors 206 may also be referred to as load cells 206 herein.

Looking to the first example force sensor configuration 500, the handle 110 comprises two load cells 206, with one load cell 206 positioned at each end of handle 110 and oriented at a same angle. The same force input would thus control either vertical or horizontal motion as each would be enabled separately. The angle of the load cell 206 may further be set to minimize any vertical force effect on the drive motion.

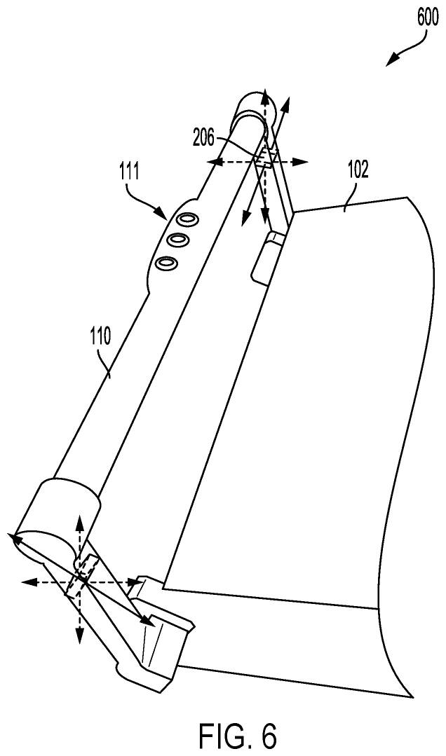

Turning now to a second example force sensor configuration 600 at FIG. 6, the second example force sensor configuration 600 uses two load cells 206 oriented at the same angle but with opposite directions. In this case the load cells 206 are oriented at equal but opposite angles. As a result, the direction of the force can be determined by determining each load cell magnitude and direction. This allows for at least partial differentiation between vertical and horizontal force inputs.

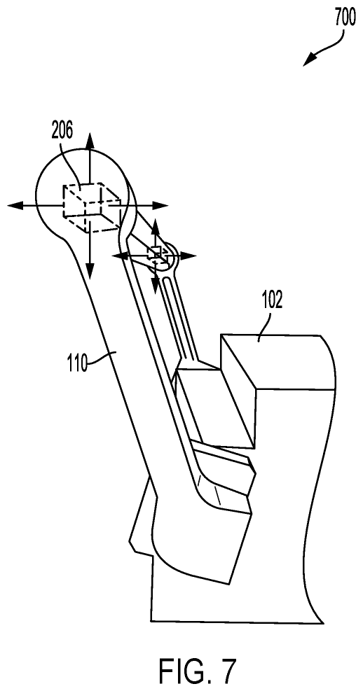

Moving to a third example force sensor configuration 700 at FIG. 7, the third example force sensor configuration 700 comprises multi-axis load cells 206 used to isolate vertical, horizontal forces as well as torque in the handle 110. That is, each of the load cells 206 may be a multi-axis load cell 206 included in the handle 110.

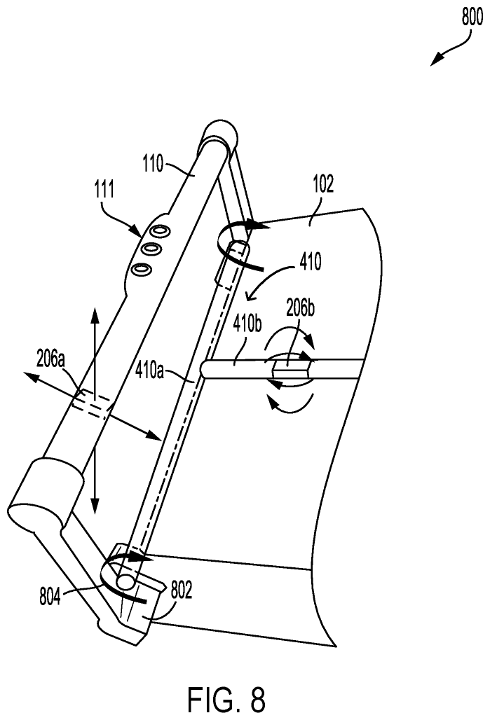

As to FIG. 8, a fourth example force sensor configuration 800 is shown, the fourth example force sensor configuration 800 comprising a mechanical linkage 410 and two or more load cells 206a, 206b to isolate vertical and horizontal motion. The mechanical linkage 410 may be a T-shaped linkage, where a top 410a of the t-shape extends parallel to the handle 110 of the patient table 102, and a base 410b of the t-shape extends perpendicularly to the handle 110. The handle 110 of the patient table 102 may be configured to mechanically pivot at a handle base 802 as indicated by arrow 804, where the handle base 802 is positioned below the top 410a of the t-shaped mechanical linkage 410 to isolate a vertical force. In this way, a first load cell 206a in the handle 110 is configured to only detect a vertical force and a second load cell 206b positioned in the mechanical linkage 410 is configured to detect forward and backward force.

Thus, provided herein are systems and methods allowing for motor assistance along the vertical and the horizontal axis of a patient table that may be controlled via a user force input to a handle of the table. By limiting movement of the patient table to only provide lifting and lowering motor assistance when in the docked state, and by further limiting movement of the patient table to only provide driving motor assistance when in the undocked state, technical advantages of providing directionally accurate motor assistance in a manner that is intuitive and efficient for a user results. Furthermore, in examples where the patient table may be transitioned from the docked to the undocked state by simply pulling on the handle, even further efficiencies may result for positioning a patient as desired.

The disclosure also provides support for a system, comprising: a patient table, an imaging system, and a controller including non-transitory instructions stored in memory executable to: adjust a drive wheel output of the patient table responsive to a user force input when undocked from the imaging system, and adjust a vertical height of the patient table responsive to the user force input when docked to the imaging system. In a first example of the system, the controller further comprises instructions executable to: not adjust the drive wheel output responsive to the user force input when docked, and not adjust the vertical height of the patient table responsive to the user force input when undocked. In a second example of the system, optionally including the first example, a drive motor is coupled to a drive wheel of the patient table, and wherein the controller further comprises instructions executable to: actuate the drive motor to adjust the drive wheel output of the patient table responsive to the user force input when undocked from the imaging system. In a third example of the system, optionally including one or both of the first and second examples, a lift motor is coupled to a support of the patient table, and wherein the controller further comprises instructions executable to: actuate the lift motor to adjust the vertical height of the patient table responsive to the user force input when docked to the imaging system. In a fourth example of the system, optionally including one or more or each of the first through third examples, a lift motor is coupled to a support of the patient table, and wherein the lift motor is configured to provide motor assistance to vertically adjust the patient table in an upward direction or a downward direction. In a fifth example of the system, optionally including one or more or each of the first through fourth examples, drive motor is coupled to a drive wheel of the patient table, and wherein the drive wheel is configured to adjust the drive wheel output in a forward direction or a reverse direction.

The disclosure also provides support for a method for controlling a motion-assisted patient table, comprising: detecting the motion-assisted patient table is in a docked state, and responsive to detecting the motion-assisted patient table is in the docked state, disabling drive assistance for the motion-assisted patient table while enabling vertical assistance for the motion-assisted patient table. In examples of the method, the method further comprises: while the motion-assisted patient table is in the docked state, receiving a request for drive assistance and not providing the drive assistance responsive to the request. In a third example of the method, optionally including one or both of the first and second examples, detecting the motion-assisted patient table is in the docked state includes directing power to a lift motor used for the vertical assistance and not directing power to a drive motor used for the drive assistance, wherein a power source for the motion-assisted patient table comprises only a single battery. In a fourth example of the method, optionally including one or more or each of the first through third examples, the method further comprises: detecting the motion-assisted patient table is in an undocked state, responsive to detecting the motion-assisted patient table is in the undocked state, disabling the vertical assistance for the motion-assisted patient table while enabling the drive assistance for the motion-assisted patient table, receiving a drive assistance request while the motion-assisted patient table is in the undocked state, and responsive to the drive assistance request while the motion-assisted patient table is in the undocked state, actuating a drive motor of the motion-assisted patient table to provide table pushing assistance or table pulling assistance. In a fifth example of the method, optionally including one or more or each of the first through fourth examples, the vertical assistance for the motion-assisted patient table is provided responsive to detecting a user input at handle of the motion-assisted patient table, and wherein providing the vertical assistance includes operating a lift motor to apply torque to assist in lifting or lowering the motion-assisted patient table responsive to the user input while the motion-assisted patient table is docked. In a sixth example of the method, optionally including one or more or each of the first through fifth examples, enabling the vertical assistance for the motion-assisted patient table includes directing power from a power source to a lift motor of the motion-assisted patient table, and wherein the motion-assisted patient table comprises only a single battery as the power source. In a seventh example of the method, optionally including one or more or each of the first through sixth examples, the method further comprises: detecting the motion-assisted patient table is in an undocked state and a brake of the motion-assisted patient table is disengaged, responsive to detecting the motion-assisted patient table is in the undocked state and the brake of the motion-assisted patient table is disengaged, disabling the vertical assistance for the motion-assisted patient table while enabling drive assistance for the motion-assisted patient table, receiving a user brake request while the motion-assisted patient table is in the undocked state with the brake disengaged, and responsive to receiving the user brake request while the motion-assisted patient table is in the undocked state, engaging the brake of the motion-assisted patient table, preventing the drive assistance for the motion-assisted patient table, and enabling the vertical assistance for the motion-assisted patient table. In an eighth example of the method, optionally including one or more or each of the first through seventh examples, the method further comprises: detecting a request to undock the motion-assisted patient table, and responsive to detecting the request to undock the motion-assisted patient table, releasing the motion-assisted patient table from a dock, and enabling drive assistance for the motion-assisted patient table while disabling vertical assistance for the motion-assisted patient table. In a ninth example of the method, optionally including one or more or each of the first through eighth examples, the method further comprises: receiving a vertical assistance request while the motion-assisted patient table is in the docked state, and actuating a lift motor of the motion-assisted patient table to provide table lifting or table lowering assistance responsive to the vertical assistance request.

The disclosure also provides support for a system for controlling a motion-assisted patient table, comprising: a table top, a support coupling the table top to a base, one or more wheels coupled to the base, a drive motor configured to provide torque to the one or more wheels, a lift motor configured to extend and retract the support, a single battery coupled to the drive motor and the lift motor, a brake, a plurality of force sensors, and a controller comprising instructions in non-transitory memory, the instructions executable to: in a first condition, responsive to detecting that the motion-assisted patient table is in a docked state, disable the drive motor, and enable the lift motor, in a second condition, responsive to an indication that the motion-assisted patient table is in an undocked state and detecting that the brake is disengaged, enable the drive motor, and disable the lift motor, and in a third condition, responsive to the indication that the motion-assisted patient table is in the undocked state and detecting that the brake is engaged, disable the drive motor, and enable the lift motor. In a first example of the system, the motion-assisted patient table is detected to be in the docked state responsive to determining a docking latch of the motion-assisted patient table is in an engaged position, and wherein the motion-assisted patient table is detected to be in the undocked state responsive to determining the docking latch of the motion-assisted patient table is in a disengaged position. In a second example of the system, optionally including the first example, the motion-assisted patient table is docked to a medical scanner in the docked state, and wherein the motion-assisted patient table is detected to be in the docked state based on an output received from the medical scanner. In a third example of the system, optionally including one or both of the first and second examples the instructions further executable to: transition the motion-assisted patient table from the docked state in first condition to the undocked state in the second condition responsive to a user input, wherein the motion-assisted patient table is docked to a medical scanner in the docked state, and wherein undocking the motion-assisted patient table includes outputting a signal at both the motion-assisted patient table and the medical scanner that the motion-assisted patient table is in the undocked state. In another representation, it is noted that alternative sensors to force sensors may be used in the handle. For example, Hall effect sensors may be implemented.

FIGS. 1. 2A, 2B, and 5-8 show example configurations with relative positioning of the various components. Unless otherwise noted, if shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

The control methods and routines disclosed herein may be stored as executable instructions in non-transitory memory and may be carried out by the control system including the controller in combination with the various sensors, actuators, and other hardware.

It will be appreciated that the configurations and routines disclosed herein are exemplary in nature, and that these specific embodiments are not to be considered in a limiting sense, because numerous variations are possible. Moreover, unless explicitly stated to the contrary, the terms “first,” “second,” “third,” and the like are not intended to denote any order, position, quantity, or importance, but rather are used merely as labels to distinguish one element from another. The subject matter of the present disclosure includes all novel and non-obvious combinations and sub-combinations of the various systems and configurations, and other features, functions, and/or properties disclosed herein.

As used herein, the term “approximately” is construed to mean plus or minus five percent of the range unless otherwise specified.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A system, comprising:
a patient table;
an imaging system; and
a controller including non-transitory instructions stored in memory executable to:
adjust a drive wheel output of the patient table responsive to a user force input when undocked from the imaging system; and
adjust a vertical height of the patient table responsive to the user force input when docked to the imaging system.

2. The system of claim 1, wherein the controller further comprises instructions executable to:
not adjust the drive wheel output responsive to the user force input when docked; and
not adjust the vertical height of the patient table responsive to the user force input when undocked.

3. The system of claim 1, wherein a drive motor is coupled to a drive wheel of the patient table, and wherein the controller further comprises instructions executable to:
actuate the drive motor to adjust the drive wheel output of the patient table responsive to the user force input when undocked from the imaging system.

4. The system of claim 1, wherein a lift motor is coupled to a support of the patient table, and wherein the controller further comprises instructions executable to:
actuate the lift motor to adjust the vertical height of the patient table responsive to the user force input when docked to the imaging system.

5. The system of claim 1, wherein a lift motor is coupled to a support of the patient table, and wherein the lift motor is configured to provide motor assistance to vertically adjust the patient table in an upward direction or a downward direction.

6. The system of claim 1, wherein drive motor is coupled to a drive wheel of the patient table, and wherein the drive wheel is configured to adjust the drive wheel output in a forward direction or a reverse direction.

7. A method for controlling a motion-assisted patient table, comprising:
detecting the motion-assisted patient table is in a docked state; and
responsive to detecting the motion-assisted patient table is in the docked state, disabling drive assistance for the motion-assisted patient table while enabling vertical assistance for the motion-assisted patient table.

8. The method of claim 7, wherein a brake of the motion-assisted patient table is engaged responsive to a user input.

9. The method of claim 7, further comprising, while the motion-assisted patient table is in the docked state, receiving a request for drive assistance and not providing the drive assistance responsive to the request.

10. The method of claim 7, wherein detecting the motion-assisted patient table is in the docked state includes directing power to a lift motor used for the vertical assistance and not directing power to a drive motor used for the drive assistance.

11. The method of claim 7, further comprising:
detecting the motion-assisted patient table is in an undocked state;
responsive to detecting the motion-assisted patient table is in the undocked state, disabling the vertical assistance for the motion-assisted patient table while enabling the drive assistance for the motion-assisted patient table;
receiving a drive assistance request while the motion-assisted patient table is in the undocked state; and
responsive to the drive assistance request while the motion-assisted patient table is in the undocked state, actuating a drive motor of the motion-assisted patient table to provide table pushing assistance or table pulling assistance.

12. The method of claim 7, wherein the vertical assistance for the motion-assisted patient table is provided responsive to detecting a user input at handle of the motion-assisted patient table, and wherein providing the vertical assistance includes operating a lift motor to apply torque to assist in lifting or lowering the motion-assisted patient table responsive to the user input while the motion-assisted patient table is docked.

13. The method of claim 7, wherein enabling the vertical assistance for the motion-assisted patient table includes directing power from a power source to a lift motor of the motion-assisted patient table.

14. The method of claim 7, further comprising:
detecting the motion-assisted patient table is in an undocked state and a brake of the motion-assisted patient table is disengaged;
responsive to detecting the motion-assisted patient table is in the undocked state and the brake of the motion-assisted patient table is disengaged, disabling the vertical assistance for the motion-assisted patient table while enabling drive assistance for the motion-assisted patient table;
receiving a user brake request while the motion-assisted patient table is in the undocked state with the brake disengaged; and
responsive to receiving the user brake request while the motion-assisted patient table is in the undocked state, engaging the brake of the motion-assisted patient table, preventing the drive assistance for the motion-assisted patient table, and enabling the vertical assistance for the motion-assisted patient table.

15. The method of claim 7, further comprising:
detecting a request to undock the motion-assisted patient table; and
responsive to detecting the request to undock the motion-assisted patient table, releasing the motion-assisted patient table from a dock, and enabling drive assistance for the motion-assisted patient table while disabling vertical assistance for the motion-assisted patient table.

16. The method of claim 7, further comprising:
receiving a vertical assistance request while the motion-assisted patient table is in the docked state; and
actuating a lift motor of the motion-assisted patient table to provide table lifting or table lowering assistance responsive to the vertical assistance request.

17. A system for controlling a motion-assisted patient table, comprising:
a table top;
a support coupling the table top to a base;
one or more wheels coupled to the base;
a drive motor configured to provide torque to the one or more wheels;
a lift motor configured to extend and retract the support;
a single battery coupled to the drive motor and the lift motor;
a brake;
a plurality of force sensors; and
a controller comprising instructions in non-transitory memory, the instructions executable to:
in a first condition, responsive to detecting that the motion-assisted patient table is in a docked state, disable the drive motor, and enable the lift motor;
in a second condition, responsive to an indication that the motion-assisted patient table is in an undocked state and detecting that the brake is disengaged, enable the drive motor, and disable the lift motor; and
in a third condition, responsive to the indication that the motion-assisted patient table is in the undocked state and detecting that the brake is engaged, disable the drive motor, and enable the lift motor.

18. The system of claim 17, wherein the motion-assisted patient table is detected to be in the docked state responsive to determining a docking latch of the motion-assisted patient table is in an engaged position, and wherein the motion-assisted patient table is detected to be in the undocked state responsive to determining the docking latch of the motion-assisted patient table is in a disengaged position.

19. The system of claim 17, wherein the motion-assisted patient table is docked to a medical scanner in the docked state, and wherein the motion-assisted patient table is detected to be in the docked state based on an output received from the medical scanner.

20. The system of claim 17, the instructions further executable to:
in a fourth condition, simultaneously allow vertical adjustment via the lift motor and drive assistance via the drive motor responsive to a user force input.
